(19) United States
(12) Patent Application Publication
(10) Pub. No.: US 2013/0315969 A1
(43) Pub. Date: Nov. 28, 2013

(54) Medicament and System for the Percutaneous Preparation of Medicaments

(71) Applicant: Meddrop Technology AG, Thundorf (CH)

(72) Inventors: Doris Barnikol-Keuten, Nieder-Olm (DE); Dieter Gulik, Reinbek-Ohe (DE)

(73) Assignee: Meddrop Technology AG, Thundorf (CH)

(21) Appl. No.: 13/959,506

(22) Filed: Aug. 5, 2013

Related U.S. Application Data
(63) Continuation of application No. 11/665,209, filed on Oct. 9, 2007.

(30) Foreign Application Priority Data
Oct. 12, 2004 (DE) 1020040495742

(51) Int. Cl. A61K 9/107

(52) U.S. Cl. CPC A61K 9/1075
USPC 424/400

(57) ABSTRACT

The invention relates to an application system provided with a micro emulsion (14) containing a medication, said micro emulsion being contained in a medicament reservoir (12), a first gas connection (18) to which oxygen can be guided, a nozzle head (28) comprising recesses (29) which are arranged on the end of the medicament reservoir (12) and an atomising nozzle (30) which is arranged in the nozzle head (28). Pressure exerted on the microemulsion for atomising as well as the microemulsion emerging therefrom atomises the oxygen into drops by means of a Venturi arrangement in the atomising nozzle (30).

### RELATED APPLICATIONS

This application is a continuation of U.S. application Ser. No. 11/665,209, filed Oct. 9, 2007, which is the U.S. National Stage of International Application No. PCT/EP2005/010909, filed on Oct. 11, 2005, published in German, which claims priority under 35 U.S.C. §119 or 365 to German Application No. DE 10 2004 049 574.2, filed Oct. 12, 2004. The entire teachings of the above applications are incorporated herein by reference.

### BACKGROUND

On administering medicinal substances as active substance materials to a patient, a balance is always to be obtained, with the dosage, between desired action and undesired side effects on the body. It is accordingly desirable to bring the medicinal substance as directly as possible to the site of action, in order accordingly to be able to work with minimum total dosages and to place the least possible burden on the body of the patient, and still to achieve the necessary active level at the site of action. This can be achieved by percutaneous administration of medicinal substances.

The skin, in particular the upper horny layer, represents though a barrier which can be overcome only with difficulty. This applies in particular for water-soluble or sparingly soluble medicinal substances.

A conventional process for the percutaneous administration of medicinal substances is the application of ointments, creams or gels to the skin. In order to improve the permeation of the active substances, use is made of “penetration promoters”, such as sulfoxides, alcohols, fatty acids, anoids, fusids, and many others. These substances reduce the resistance to penetration of the horny layer and facilitate the permeation of the medicinal substances.

Dosing possibilities which are only approximate are disadvantages of this process. Because of this, the content of medicinal substances in the preparations has to be kept low as a precaution, resulting in the desired high active level not being reached even in the target tissues. Moreover, despite the use of penetration promoters, the depth of penetration of conventional preparations is only very low.

Furthermore, different methods are known for overcoming the barrier of the skin (compare Müller/Hildebrand; Pharmazeutische Technologie: Moderne Arzneiformen [Pharmaceutical Technology: Modern medicinal forms], ISBN 3-8047-1549-4, chapter 13).

In particular, transdermal therapeutic systems (TTS) have been developed. TTSs are technical devices which are placed on a specific area of the skin in an adherent fashion and which deliver, by diffusion through the skin, to the body a specific dose of the medicinal substance according to different mechanisms with a specific time-related feed. The objective in this connection is in particular a systemic action with a defined profile of the active level. In order to accelerate the permeation of the medicinal substance into the skin, TTS systems also have ultrasound heads or electrodes, in order to deliver current impulses to the skin and accordingly to promote pore formation in the skin by mechanical or electrical stimuli.

A disadvantage here is that a targeted local application by means of TTS is not possible. There is the fact that not all medicinal substances can be administered by diffusion. This applies in particular for water-soluble and sparingly soluble medicinal substances.

Furthermore, medicinal substances are applied to the skin in microemulsions. Because of the low surface tension and large interface in the microemulsion, water-soluble, fat-soluble and sparingly soluble medicinal substances can be dispersed therein. With the help of a microemulsion, success is achieved in introducing the medicinal substances into the horny layer of the skin (stratum corneum) within a short time.

Even with the help of microemulsions, alone, success is not satisfactorily achieved, though, in temporarily abolishing the barrier function of the skin to the desired extent and in applying all kinds of medicinal substances through the skin.

It is an object of the invention to remedy the abovementioned disadvantages of the state of the art.

Specifically, it is an object of the invention to make available preparations (subsequently referred to as medicaments) which satisfactorily penetrate the barrier of the skin.

Furthermore, it is an object of the invention to make available a system with which it is possible, on any area of the skin, to penetrate the barrier of the skin and to percutaneously apply an active substance or a combination of active substances.

Furthermore, it is an object of the invention to make available a system with which the medicinal substances to be applied can be accurately dosed.

It is an additional object of the invention to apply the maximum daily dose locally.

It has been found, surprisingly, that this and additional unmentioned objects are achieved with the help of a system according to the invention for the percutaneous administration of medicinal substances, exhibiting a microemulsion, into which the medicinal substances are introduced, and a device for the atomization of the microemulsion, preferably in an oxygen-comprising atmosphere (the term “atomization” is to be understood here as the fine dispersing of liquid using a propellant gas).

Furthermore, these objects are achieved by a microemulsion enriched with oxygen which comprises at least one medicinal substance for percutaneous administration.

A microemulsion for the percutaneous administration of medicinal substances which exhibit medicinal substances for the improved supply of oxygen to the skin also achieve the object according to the invention.

The combination of the various mechanisms of the novel process can result in significant synergistic effects in the permeation of active substances into the skin, as is explained subsequently.

Through the extraordinarily small droplets of the high performance atomizer, the microemulsion charged with active substance is applied to the skin in finely divided form. Because of the low surface tension of the microemulsion, a huge spreading effect arises in this connection. The horny layer of the skin and the microemulsion have similar upper structures, such as lamellae or tubuli, formed from bilipid layers. These upper structures of the horny layer contribute crucially to the resistance to permeation of this layer. The finely dispersed application of the droplets presumably results in “fusion” of the microemulsion with the horny layer according to the principle “similia similibus”. As a result of the fusion, the abovementioned upper structures dissolve and the active substances can diffuse into the skin at a reinforced level. The use of oxygen as propellant gas results in the lipid-comprising droplets of the atomizer being enriched in oxygen. This oxygen is, like the active substances, introduced into the skin layer, which results in an increase in the oxygen partial pressure in the skin. This elevated partial pressure strongly stimulates the microcirculatory flow. Through this, the active substance materials which have diffused in are more strongly entrained convectively inward into the tissue.

The combined use of microemulsions, fine droplets and oxygen in the process according to the invention also results in an increase in the permeation of active substances in three successive steps:

1. The microemulsion and accordingly the active substances are very finely divided and spread over the surface of the skin.

2. The horny layer barrier is overcome and

3. The microcirculatory transport through the skin is increased, namely first by the high performance atomization, secondly by the microemulsion and thirdly by the oxygen.

### DETAILED DESCRIPTION OF THE INVENTION

The skin is the biggest organ in the body and closes off the outside. It has, in its operation, to perform a number of tasks.

In first place is the protective function against mechanical effects, such as impacts, pressure or rubbing, and against the penetration of bacteria, viruses and fungi through an acidic sheathing. Furthermore, the skin protects against heat, cold, light and harmful substances.

The skin is also a sense organ: special sensors detect pressure, temperature, pain and itching.

The skin also intervenes, by regulation of the water and heat budget, in a regulating fashion in the function of the whole body.

In broad terms, the skin consists of three layers: of the subcutis, of the corium (dermis) and of the epidermis.

The subcutis consists of fat, large blood vessels, glands and small muscles. It serves, e.g., as “larder” and for the damping of mechanical effects. The dermis, with its collagen and elastomer fibers, brings about hold and elasticity of the skin and accordingly also resistance to tearing. Sensory cells (sensors) for reception of the abovementioned sensations are also located in the dermis. It comprises much hyaluronic acid and chondroitin sulfate, thus glucosaminoglucans, which make possible, as reversible gels, the transport of biological molecules and cytotaxis.

The epidermis is of particular importance and particular interest in closing off the body from the outside since this layer altogether guarantees the integrity of the skin, the very outermost layer, the horny layer, playing a crucial role.

This layer consists of a layer, approximately 10 cells thick, of keratinized, i.e. dead, flat cells (horn cells, stratum corneum); it is divided up yet further into an upper loose layer (stratum disjunctum) and into a lower firmer layer, the stratum conjunctum. The horn cells are constantly peeling off toward the outside and are produced by division in the “stratum germinativum”, the germinative layer, located thereunder.

The particular microstructure of the stratum corneum consists of flat, brick-like keratinized cells (corneocytes). The intracellular matrix is particularly structured. It consists, approximately parallel to the skin surface, of lipoid bilayers: in the stratum corneum, approximately one hundred aqueous and lipid phases alternate. In the formulation sense, the horny layer represents a “water-in-oil emulsion” in the form of a lamellar bilayer. This constantly regenerating layer, with a thickness of only approximately 12 μm, forms, with the help of its complex two-phase upper structures, secure protection for the cells of the stratum germinativum located thereunder: without the horny layer, a “wound bed” is produced.

The horny layer of the skin is of particular importance for closing off from the outside, especially in its barrier function. This is the case with regard to the density, the oxygen partial pressure (PO₂), the pH and the water content.

The barrier for hydrogen ions, which form an acidic protective sheathing, is particularly important. Equally important is a barrier for oxygen, by putting up great resistance to the diffusion of this. This results in a decrease in the oxygen partial pressure of the air from 150 torr to approximately 50 torr. Accordingly, the vital cells of the skin epithelium of the intact skin are protected from an excessively high oxidatively damaging oxygen partial pressure.

So advantageous the effective barrier function in the horny layer is for the body, so disadvantageous it proves to be for transdermal transport of medicinal substances. In such cases, the corneal barrier has to be temporarily abolished.

It has been found, surprisingly, that the barrier function of the skin, by introduction of oxygen into the horny layer and accordingly the increase in the oxygen partial pressure on the tissue side of the stratum corneum, results in an improved transdermal transport of medicinal substances.

The transmembrane pressure of the oxygen is increased by the increase in the oxygen partial pressure on the tissue side of the stratum corneum, which is presumably a reason for the improved transdermal transport of medicinal substances.

Because of the above described lamellar structure of alternating water and oil phases in the stratum corneum, microemulsions can be particularly suitably introduced into the stratum corneum (compare Müller/Hildebrand; Pharmazeutische Technologie: Moderne Arzneiformen [Pharmaceutical Technology: Modern medicinal forms], ISBN 3-8047-1549-4, chapter 15). In a preferred embodiment of the invention, these are used as vehicle systems for oxygen or medicinal substances and also base materials for medicaments.

Such microemulsions are known and are used in cosmetics and the pharmaceutical industry. These are available commercially, for example under the trade name “Nanoemulsion” from Sangui AG.

Microemulsions within the meaning of the invention are thermodynamically stable systems which exhibit at least water, surfactants and lipid. The term “a surfactant” is understood to mean emulsifiers which can be ionic or nonionic. Examples of surfactants which can be used are known under the trade name Tween, Span and Synperonic PEL 101.

Lipids which can be used are fatty oils or mineral oils, for example isopropyl myristate and isopropyl palmitate.

Microemulsions which can be used in the context of this invention can be oil-in-water microemulsions or water-in-oil microemulsions. In this connection, oil droplets in a water matrix or water droplets in an oil matrix are formed.

Such microemulsions exhibit droplet sizes in the range from 10 nm to 1 μm, preferably from 10 nm to 500 nm, particularly preferably from 10 nm to 300 nm.

The mean droplet size of a microemulsion which can be used in the context of the invention is not limited. The mean droplet size is preferably less than 300 nm, particularly preferably less than 150 nm.

Such microemulsions preferably exhibit interfaces of more than 200 m² per ml, particularly preferably of more than 400 m² per ml and very particularly preferably of more than 600 m² per ml.

Because of the hydrophilic and lipophilic portion of the microemulsions and of the low surface tension and of the large interface, it is possible to disperse, in microemulsions, both water-soluble and fat-soluble and/or sparingly soluble medicinal substances. The choice of the surfactants is in this connection made according to the active substance and the effect desired. Ionic surfactants are generally particularly effective, while nonionic surfactants are particularly kind to the skin.

Microemulsions according to the invention relate, inter alia, to the medicinal use of liquid medicaments based on microemulsions in the therapy of pain, for the treatment of circulatory disorders and for the healing of wounds in degenerated skin, e.g. in elderly people.

Medicinal substances based on such microemulsions can, in addition to the parent substances of the microemulsion, exhibit base materials for medicaments and medicinal substances. These base materials and medicinal substances can be of natural and synthetic origin. In the context of this invention, base materials and medicinal substances of natural origin are particularly preferred, without this being limiting.

Examples of natural base materials and the effect thereof are represented in table 1. Base materials which can be used in the context of this invention are not, however, limited thereto.

TABLE 1
Natural base materials and the effect thereof

| Base materials | Effect |
|---|---|
| Aloe vera | favoring the blood flow |
| | contributing to moistness |
| | inhibiting inflammation |
| | removing wrinkles |
| | nourishing the skin |
| Arnica oil (fat) | alleviating pain |
| | inhibiting inflammation |
| | causing hyperemia |
| | favoring the blood flow |
| | healing wounds |
| Avocado oil | binding of moisture |
| | regenerating |
| | alleviating itching |
| | healing wounds |
| | nourishing the skin |
| Borage oil | skin regenerating |
| | alleviating itching |
| Centella oil | regenerating |
| | regulating connective tissue (scars) |
| | antiinflammatory |
| | healing wounds |
| Rose of Sharon oil | antiinflammatory |
| | analgesic |
| | causing hyperemia |
| | antispasmodic |
| Jojoba oil | inhibiting inflammation |
| | regenerating |
| | healing wounds |
| Corn oil | antioxidant |
| Almond oil | regenerating |
| | nourishing |
| Evening primrose oil | healing wounds |
| | antibacterial |
| | alleviating itching |
| Neem oil | antibacterial |
| | antimycotic |
| Olive oil | causing hyperemia |
| | favoring the blood flow |
| | healing wounds |
| Marigold oil | antiinflammatory |
| | antirheumatic |
| | favoring the blood flow |
| Shea butter | healing wounds |
| | regenerating |
| Grapeseed oil | astringent |
| Wheat germ oil | regenerating |
| | nourishing the skin |
| Dog rose oil | contributing to moistness |
| Rose hip oil | skin regenerating |
| | alleviating itching |
| | nourishing |
| | healing wounds |

The medicinal substances which can be used in the context of this invention are not limited. In this connection, natural and synthetic medicinal substances can be used. In the context of this invention, natural medicinal substances obtained from plants are preferred. Essential oils which can be obtained from plant parts are particularly preferred as medicinal substances. Examples of plant species and genera, inclusive of their chemotypes, which comprise essential oils in the most varied plant parts, which can be used as medicinal substances in microemulsions in the context of this invention, and also the therapeutic effect thereof in external application, are represented in table 2; however, these are not limited thereto.

TABLE 2
Plant species and genera, inclusive of their chemotypes,
which comprise essential oils in the most varied plant parts,
and also the therapeutic effect thereof in external application

| | Species/Genus/ | |
|---|---|---|
| Name | Chemotypes | Properties |
| Angelica oil | Angelica | skin regenerating |
| Valerian oil | Valeriana | diuretic |
| | | skin regenerating |
| Basil oil | Ocimum | antibacterial |
| | Chemotype | antispasmodic |
| | Methyl chavicol | antiviral |
| | | antiinflammatory |
| | | analgesic |
| | | deblocking |
| | | caring for varicose veins |
| Bay oil | Pimenta | antibacterial |
| Pimento oil | | antimycotic |
| | | antiviral |
| Mugwort oil | Artemisia | antiviral |
| Benzoin resin | Styrax | antiinflammatory |
| | | antiseptic |
| | | skin regenerating |
| | | cell renewal |
| Bergamot oil | Citrus aurantium var. | antiseptic |
| | Bergamia | epithelizing |
| | | healing wounds |
| | | skin regenerating |
| Winter savory oil | Satureja Montana | analgesic |
| | | antibacterial |
| | | antimycotic |
| | | antiseptic |
| | | immunomodulating |
| Birch oil | Betula | antiinflammatory |
| | 98% methyl salicylate | antirheumatic |
| | | antispasmodic |
| | | alleviating pain |
| | | vasodilative |
| Cajeput oil | Melaleuca | antibacterial |
| | | antiviral |
| | | caring for varicose veins |
| Cassia oil | Cinnamomum cassia | antibacterial |
| | | anticoagulant |
| | | antimycotic |
| | | antiviral |
| | | causing hyperemia |
| Cistus oil | Cistus | antibacterial |
| | | antihemorrhagic |
| | | antiviral |
| Eucalyptus oil | Eucalyptus | analgesic |
| | | antibacterial |
| | | antimycotic |
| | | antiinflammatory |
| | | antiviral |
| Fennel oil | Foeniculum | analgesic |
| | | dehydrating |
| Fir needle oil | Abies | antiinflammatory |
| | | causing hyperemia |
| Galbanum oil | Ferula | antiinflammatory |
| | | antiseptic |
| | | healing wounds |
| Geranium oil | Pelargonium graveolens | astringent |
| | | antibacterial |
| | | antimycotic |
| | | deblocking |
| | | caring for the skin |
| | | caring for varicose veins |
| | | healing wounds |
| Geranium oil | Geranium macrorrhizum | antiseptic |
| “true geranium” | | epithelizing |
| Clove oil | Eugenia caryophyllata | antibacterial |
| | | antimycotic |
| | | antiviral |
| Ho wood oil | Cinnamomum | antibacterial |
| | | antimycotic |
| | | antiviral |
| Immortelle oil | Helichrysum | analgesic |
| Everlasting oil | | anticoagulant |
| | | epithelizing |
| Ginger oil | Zingiber | analgesic |
| | | causing hyperemia |
| Blue camomile oil | Matricaria camomilla | antiinflammatory |
| | | healing wounds |
| Roman camomile | Anthemis nobilis | analgesic |
| oil | | antiinflammatory |
| Wild camomile oil | Ormensis mixta | antibacterial |
| | | antimycotic |
| | | healing wounds |
| Camphor oil | Cinnamomum | anesthetic |
| | | analgesic |
| | | antibacterial |
| | | antiinfective |
| | | antimycotic |
| | | antirheumatic |
| | | antiviral |
| | | diuretic |
| | | causing hyperemia |
| | | immunomodulating |
| | | rheumatic pain |
| | | spasmolytic |
| Pine oil | Pinus | antibacterial |
| | | causing hyperemia |
| | | protecting from edema |
| Mountain pine oil | Pinus mugo | antiinflammatory |
| | | immunomodulating |
| Lavender oil | Lavendula | analgesic |
| | | antibacterial |
| | | anticoagulant |
| | | antimycotic |
| | | antiinflammatory |
| | | epithelizing |
| | | alleviating itching |
| Spanish sage oil | Salvia | analgesic |
| | | antiinfective |
| | | antispasmodic |
| | | tonic |
| Lemongrass oil | Cymbopogon | antibacterial |
| | | antiinflammatory |
| | | antiviral |
| | | vasodilative |
| | | immunomodulating |
| Laurel oil | Laurus | analgesic |
| | | antibacterial |
| | | anticoagulant |
| | | antispasmodic |
| | | mucolytic |
| | | protecting from edema |
| Marjoram oil | Origanum | analgesic |
| | | antibacterial |
| | | antispasmodic |
| | | diuretic |
| Manuka oil | Leptospermum | antibacterial |
| | | antimycotic |
| | | antiinflammatory |
| | | antirheumatic |
| | | sedative |
| | | skin regenerating |
| | | alleviating itching |
| Melissa oil | Melissa | analgesic |
| | | antiviral |
| | | inhibiting inflammation |
| | | immunomodulating |
| | | caring for varicose veins |
| Myrrh oil | Commiphora | antibacterial |
| | | antiinflammatory |
| | | antiviral |
| | | epithelizing |
| | | skin regenerating |
| Niaouli oil | Melaleuca | analgesic |
| | | antiinfective |
| | | antimycotic |
| | | antiviral |
| | | immunomodulating |
| | | caring for varicose veins |
| Oregano oil | Origanum | analgesic |
| | | antibacterial |
| | | antimycotic |
| | | antiviral |
| | | causing hyperemia |
| | | immunomodulating |
| Patchouli oil | Pogostemon | analgesic |
| | | antiinfective |
| | | antimycotic |
| | | antiinflammatory |
| | | diuretic |
| | | deblocking |
| | | epithelizing |
| | | immunomodulating |
| Petitgrain oil | Citrus aurantium | antiinfective |
| | | antiinflammatory |
| | | antispasmodic |
| Balsam Peru oil | Myroxylon | antibacterial |
| | | antiinflammatory |
| | | antispasmodic |
| Pepper oil (black) | Piper | analgesic |
| | | antibacterial |
| | | antiviral |
| | | diuretic |
| | | causing hyperemia |
| Peppermint oil | Mentha | analgesic |
| | | anesthetizing |
| | | antibacterial |
| | | antimycotic |
| | | antiparasitic |
| | | antiviral |
| | | epithelizing |
| | | cooling |
| | | spasmolytic |
| Pimento oil | Pimenta | antibacterial |
| | | antimycotic |
| | | antiviral |
| Tansy oil | Tanacetum | analgesic |
| | | antiallergic |
| | | alleviating itching |
| | | caring for varicose veins |
| Ravensara oil | Ravensara | antibacterial |
| | | antimycotic |
| | | antiviral |
| Rose oil | Rosa damaszena | antiinflammatory |
| | | antiviral |
| | | skin regenerating |
| Rosemary oil | Rosmarinus | analgesic |
| | Chemotype “Moroccan” | diuretic |
| | Cineol | fungicidal |
| | | causing hyperemia |
| Savin oil | Juniperus | analgesic |
| | | causing hyperemia |
| Sage oil | Salvia | antibacterial |
| | | antimycotic |
| | | antiviral |
| Sandalwood oil | Santalum | deblocking |
| | | epithelizing |
| Yarrow oil | Achillea | analgesic |
| | | antiinflammatory |
| | | epithelizing |
| Black cumin oil | Nigella | analgesic |
| | | antiallergic |
| | | antiinflammatory |
| Spike lavender oil | Lavendula spica | analgesic |
| | | antiinfective |
| | | antiviral |
| | | fungicidal |
| Tagetes oil | Tagetes | antimycotic |
| Tea tree oil | Melaleuca | analgesic |
| | | antibacterial |
| | | antimycotic |
| | | antiparasitic |
| | | antiinflammatory |
| | | antiviral |
| | | epithelizing |
| | | immunomodulating |
| | | caring for varicose veins |
| Texas cedar oil | Juniperus mexicana | deblocking |
| | | diuretic |
| Thuja oil | Thuja | antiinfective |
| | | antiviral |
| | | diuretic |
| | | epithelizing |
| | | healing wounds |
| Thyme oil | Thymus vulgaris | antibacterial |
| | Chemotype Linalool and | antimycotic |
| | Geraniol | antiviral |
| | Thymus | antibacterial |
| | Chemotype Thujanol | antiviral |
| | | immunomodulating |
| | Thymus | analgesic |
| | Chemotype Thymol and | antiinfective |
| | Carvacrol | immunomodulating |
| Vetiver oil | Vetiveria | caring for the skin |
| | | causing hyperemia |
| Juniper oil | Juniperus | antibacterial |
| | | antirheumatic |
| | | diuretic |
| Frankincense oil | Boswellia | antiinflammatory |
| | | epithelizing |
| | | immunomodulating |
| Silver fir oil | Abies | antiseptic |
| | | causing hyperemia |
| Wintergreen oil | Gaultheria | antiinflammatory |
| | | antispasmodic |
| | | alleviating pain |
| | | vasodilative |
| Hyssop oil | Hyssopus | antibacterial |
| | | antiviral |
| | Hyssopus var. | antiinflammatory |
| | Decumbens | antiviral |
| Cinnamon oil | Cinnamomum verum | antibacterial |
| | | antimycotic |
| | | antiparasitic |
| | | antiviral |
| | | causing hyperemia |
| | | immunomodulating |
| Lemon oil | Citrus | astringent |
| | | antibacterial |
| | | anticoagulant |
| | | antiviral |
| | | caring for varicose veins |
| Cypress oil | Cupressus | astringent |
| | | diuretic |
| | | deblocking |
| | | caring for varicose veins |

Preferably used medicinal substances and the active properties thereof are listed in table 3. These are subdivided into essential oils, plant extracts and synthetic single substances. The medicinal substances which can be used in the context of this invention are not, though, to be limited thereto.

TABLE 3
Active properties of essential oils, plant extracts and
single substances isolated from these plant extracts

| | | | Chemically/pharmaceutically |
|---|---|---|---|
| | | | active |
| Properties | Essential oils | Plant extracts | substances |
| Astringent | Geranium oil | Tannins, e.g. | |
| | Lemon oil | Quercus | |
| | Cypress oil | Extract from Stipites | |
| | | Dulcamarae | |
| | | Hamamelis extract | |
| Acne | | | Azelain |
| | | | Tretinoin |
| | | | Isotretinoin |
| | | | Adapalene |
| | | | Benzoyl peroxide |
| Analgesic | Basil oil | Rose of Sharon oil | Carboxylic acids, e.g.: |
| | Winter savory | Fructus Capsici | Salicylic acid |
| | oil | (capsaicin) | Diflunisal |
| | Birch oil | Comfrey extract | Salicylamide |
| | Fennel oil | Symphytum extract | Ethenzamide |
| | Fir needle oil | Harpagophytum | Acetylsalicylic acid |
| | Ginger oil | Procumbens | Salsalate |
| | Roman | Willow bark | Acetic acid |
| | camomile oil | Guaiacwood | derivatives, e.g.: |
| | Camphor oil | Arnica extract | Indomethacin/ |
| | Extra lavender | | Acemetacin, |
| | oil | | Proglumetacin |
| | Spanish sage oil | | Diclofenac |
| | Laurel oil | | Tolmetin |
| | Marjoram oil | | Lonazolac |
| | Melissa oil | | Fenbufen |
| | Niaouli oil | | Aceclofenac |
| | Oregano oil | | Etofenamate |
| | Patchouli oil | | |
| | Pepper oil | | |
| | Peppermint oil | | |
| | Tansy oil | | |
| | Rosemary oil | | |
| | Savin oil | | |
| | Yarrow oil | | |
| | Spike lavender | | |
| | oil | | |
| | Tea tree oil | | |
| | Thyme oil | | |
| | Wintergreen oil | | |
| Continuation: | | | Propionic acid |
| Analgesic | | | derivatives, e.g.: |
| | | | Ibuprofen |
| | | | Ketoprofen |
| | | | Flurbiprofen |
| | | | Tiaprofenic acid |
| | | | Fenoprofen |
| | | | Naproxen |
| | | | Dexketoprofen |
| | | | Dexibuprofen |
| | | | Heterocyclic ketoenol |
| | | | acids |
| | | | Oxicams: |
| | | | Piroxicam |
| | | | Tenoxicam |
| | | | Metoxicam |
| | | | Meloxicam |
| | | | Lornoxicam |
| | | | Anthranilic acid |
| | | | derivatives: |
| | | | Mefenamic acid |
| | | | Flufenamic acid |
| | | | Niflumic acid |
| Continuation: | | | Other derivatives: |
| Analgesic | | | Nabumetone |
| | | | Azapropazone |
| | | | Aceclofenac |
| | | | Caffeine |
| | | | Pyrazolidiones: |
| | | | Azapropazone |
| | | | Oxyphenbutazone |
| | | | Phenylbutazone/ |
| | | | Mofebutazone |
| | | | Azapropazone |
| | | | Additional substance |
| | | | categories: |
| | | | Paracetamol |
| | | | Niflumic acid |
| | | | Bufexamac |
| Neuropathies | | | Pyrazolinones: |
| Neuropathic | | | Propylphenazone |
| | | | Metamizole |
| | | | Cox-2 inhibitors, e.g. |
| | | | Celecoxib |
| | | | Rofecoxib |
| | | | Valdecoxib |
| | | | Etoricoxib |
| | | | Parecoxib |
| | | | Vitamin B complex |
| | | | α-Lipoic acid |
| | | | L-Camithin |
| | | | Peripheral |
| | | | sympathetic blockers: |
| | | | Clonidine |
| | | | Homeopathic |
| | | | preparation |
| Anesthetizing | Camphor oil | | Ester local anesthetics |
| | Peppermint oil | | Benzocaine |
| | Thyme oil | | Procaine (0) |
| | | | Tetracain (0) |
| | | | Thymol |
| Continuation: | | | Amide local |
| Anesthetizing | | | anesthetics |
| | | | Prilocaine |
| | | | Mepivacaine |
| | | | Lidocaine |
| | | | Etidocaine |
| | | | Bupivacaine |
| | | | Levobupivacaine |
| | | | Ropivacaine |
| | | | Articaine |
| | | | Fomocaine |
| Antiallergic | Black cumin oil | | Glucocorticoids |
| Antibacterial | Bay oil | Evening primrose oil | Urea |
| Antiinfective | Winter savory | Neem oil | Thymol |
| | oil | Extracts from Stipites | Chlorhexidine |
| | Cajeput oil | Dulcamarae | Antibiotics: |
| | Cassia oil | | Fusidic acid |
| | Cistus oil | | Mupirocin |
| | Eucalyptus oil | | Sulfadiazine |
| | Geranium oil | | Erythromycin |
| | Clove oil | | Clindamycin |
| | Ho wood oil | | Tetracycline |
| | Camomile oil | | Medocycline |
| | Camphor oil | | |
| | Pine oil | | |
| | Garlic oil | | |
| | Lavender oil | | Tyrothricin |
| | Extra lavender | | Gentamycin |
| | oil | | Neomycin |
| | Spanish sage oil | | Bacitracin |
| | Lemongrass oil | | Chloramphenicol |
| | Marjoram oil | | Polymyxin |
| | Manuka oil | | Kanamycin |
| | Carnation oil | | |
| | Niaouli oil | | |
| | Oregano oil | | |
| | Patchouli oil | | |
| | Balsam Peru oil | | |
| | Petitgrain oil | | |
| | Peppermint oil | | |
| | Black pepper oil | | |
| | Pimento oil | | |
| | Sage oil | | |
| | Spike lavender | | |
| | oil | | |
| | Tea tree oil | | |
| | Thuja oil | | |
| | Thyme oil | | |
| | Juniper oil | | |
| | Hyssop oil | | |
| | Cinnamon oil | | |
| | Lemon oil | | |
| Anti- | Cistus oil | Hamamelis extract | |
| hemorrhagic | | | |
| Antihistaminic | Black cumin oil | | Glucocorticoids |
| Anti- | | Sage | Camphoric acid |
| hyperhydrotic | | Walnut leaves | Methenamine |
| | | Oak bark | Aluminum chlorate |
| | | Tannins, e.g. oak bark | hexahydrate |
| Anticoagulant | Immortelle oil | | Hirudin |
| | Everlasting oil | | Hirudin derivatives |
| | Cinnamon oil | | Heparins, in particular |
| | Lavender oil | | also low molecular |
| | Laurel oil | | weight |
| | Lemon oil | | |
| Antimycotic | Bay oil | Neem oil | Azole derivatives: |
| | Pimento oil | Extracts from Stipites | Clotrimazole |
| | Winter savory | Dulcamarae | Bifonazole |
| | oil | | Econazole |
| | Cassia oil | | Fenticonazole |
| | Eucalyptus oil | | Isoconazole |
| | Geranium oil | | Oxiconazole |
| | Clove oil | | Sertaconazole |
| | Ho wood oil | | Tioconazole |
| | Camphor oil | | Miconazole |
| | Cinnamon oil | | Ketoconazole |
| | Lavender oil | | Itraconazole |
| | Extra lavender | | Fluconazole |
| | oil | | Voriconazole |
| | Manuka oil | | Sertaconazole |
| | Carnation oil | | |
| | Niaouli oil | | |
| | Oregano oil | | |
| | Patchouli oil | | |
| | Peppermint oil | | |
| Continuation: | Pimento oil | | Squalene epoxidase |
| Antimycotic | Rosemary oil | | inhibitors, e.g: |
| | Sage oil | | Terbinafin |
| | Spike lavender | | Naftifin |
| | oil | | Morpholines, e.g.: |
| | Tagetes oil | | Amorolfin |
| | Tea tree oil | | Other antimycotically |
| | Thyme oil | | effective substances, |
| | | | e.g.: |
| | | | Amphotericin B |
| | | | Griseofulvin |
| | | | Flucytosin |
| | | | Ciclopirox |
| | | | Nystatin |
| | | | Natamycin |
| | | | Thiocarbonates |
| Combating | Valerian oil | Aesculus | |
| edema | Basil oil | hippocastanum | |
| Diuretic | Fennel oil | Ruscus aculeatus | |
| Deblocking | Geranium oil | Melilotus officinalis | |
| Dehydrating | Camphor oil | Fagopyrum | |
| Protecting from | Pine oil | esculentum | |
| edema | Laurel oil | Red vine leaf extract | |
| | Marjoram oil | Solidago virgaurea | |
| | Patchouli oil | Stinging nettle | |
| | Pepper oil | | |
| | Rosemary oil | | |
| | Sandalwood oil | | |
| | Black cumin oil | | |
| | Texas cedar oil | | |
| | Thuja oil | | |
| | Juniper oil | | |
| | Cypress oil | | |
| Antioxidant | | Flavonoids | Selenium |
| | | Anthocyans | Manganese |
| | | Proanthoxycyanidines | Copper |
| | | Carotenoids | L-Glutathione |
| | | β-carotene: | L-Cysteine |
| | | Lycopene | Coenzyme Q₁₀ |
| | | Zeaxanthin | α-Lipoic acid |
| | | Vitamin A, C and E | |
| Antiparasitic | Peppermint oil | Neem oil | Crotamiton |
| | Cinnamon oil | | Permethrin |
| | Tea tree oil | | Benzyl benzoate |
| | | | Allethrin |
| Anti- | Basil oil | Melilotus officinalis | Steroidal anti- |
| inflammatory | Benzoin resin | Ruscus aculeatus | inflammatories, such |
| | Birch oil | Aesculus | as glucocorticoids |
| | Camphor oil | hippocastanum | Bufexamac |
| | Eucalyptus oil | Rose of Sharon oil | Glycyrrhetinic acid |
| | Fir needle oil | Marigold | Thymol |
| | Galbanum oil | Aloe vera | Cavacrol |
| | Rose of Sharon | Jojoba | Camphor |
| | oil | Evening primrose oil | Eugenol |
| | Blue camomile | Borage oil | Cinnamaldehyde |
| | oil | Cardiospermum | Capsaicin |
| | Roman | halicacabum | |
| | camomile oil | Tannins, e.g. from | |
| | Mountain pine | Quercus and | |
| | oil | Synthetica | |
| | Lavender oil | | |
| | Extra lavender | | |
| | oil | | |
| | Lemongrass oil | | |
| Continuation: | Manuka oil | Extracts from Stipites | |
| Anti- | Myrrh oil | Dulcamarae | |
| inflammatory | Carnation oil | Symphytum extracts | |
| | Patchouli oil | Hamamelis extract | |
| | Petitgrain oil | Camomile | |
| | Balsam Peru oil | Arnica oil | |
| | Rosemary oil | Propolis | |
| | Yarrow oil | | |
| | Black cumin oil | | |
| | Ledum palustre | | |
| | oil | | |
| | Tea tree oil | | |
| | Thyme oil | | |
| | Frankincense oil | | |
| | Wintergreen oil | | |
| | Hyssop oil | | |
| | Cinnamon oil | | |
| Antirheumatic | Birch oil | Fructus Capsici | See list analgesically |
| | Camphor oil | Capsaicin | chemically/ |
| | Manuka oil | Nicotinic acid | pharmaceutically |
| | Rosemary oil | Salicylate | effective substances |
| | Juniper oil | Cortex Salicis | Salicin |
| | Wintergreen oil | Urtica dioica | |
| | Cinnamon oil | Urtica urens | |
| Antiseptic | Benzoin resin | | |
| | Bergamot oil | | |
| | Winter savory | | |
| | oil | | |
| | Galbanum oil | | |
| | Geranium oil | | |
| | Camphor oil | | |
| | Silver fir oil | | |
| Antispasmodic | Basil oil | | |
| | Birch oil | | |
| | Spanish sage oil | | |
| | Laurel oil | | |
| | Marjoram oil | | |
| | Balsam Peru oil | | |
| | Petitgrain oil | | |
| | Wintergreen oil | | |
| Antiviral | Basil oil | Extractum | Aciclovir/ |
| | Bay oil | podophyllum | Valciclovir |
| | Pimento oil | (podophyllin) | Penciclovir/ |
| | Cajeput oil | Extractum Melissae | Famciclovir |
| | Cassia oil | Fructus Capsici | Idoxuridine/ |
| | Cistus oil | Capsaicin | Bivudine |
| | Eucalyptus oil | | Trifluridine |
| | Clove oil | | Vidarabine |
| | Ho wood oil | | Tromantadine |
| | Camphor oil | | Foscarnet |
| | Cinnamon oil | | Interferon-β |
| | Lemongrass oil | | Podophyllotoxin |
| | Melissa oil | | |
| | Myrrh oil | | |
| | Niaouli oil | | |
| | Oregano oil | | |
| | Pepper oil | | |
| | Peppermint oil | | |
| | Pimento oil | | |
| | Sage oil | | |
| | Spike lavender | | |
| | oil | | |
| | Tea tree oil | | |
| | Thuja oil | | |
| | Thyme oil | | |
| | Hyssop oil | | |
| | Lemon oil | | |
| Regenerating | | Allium cepa | Heparin |
| connective | | Centella asiatica | Asiaticoside |
| tissue | | | |
| Erectile | | | Alprostadil (PGE 1) |
| dysfunction | | | Sildenafil citrate |
| | | | Vardenafil |
| | | | Tadalafil |
| | | | Favoring the blood |
| | | | flow, such as benzyl |
| | | | nicotinate |
| Epithelizing | Bergamot oil | | |
| | Geranium oil | | |
| | Immortelle oil | | |
| | Everlasting oil | | |
| | Lavender oil | | |
| | Extra lavender | | |
| | oil | | |
| | Myrrh oil | | |
| | Patchouli oil | | |
| | Peppermint oil | | |
| | Sandalwood oil | | |
| | Yarrow oil | | |
| | Tea tree oil | | |
| | Thuja oil | | |
| | Frankincense oil | | |
| Binding of | | Avocado oil | Urea |
| moisture | | Dog rose | Glycerol |
| | | Aloe vera | Glycine |
| Vasodilative | Lemongrass oil | | Nitro preparations |
| Hair loss | | | Finasteride |
| | | | Minoxidil |
| Nourishing the | Angelica oil | Dog rose | Amino acids |
| skin | Valerian oil | Almond oil | Vitamins |
| Caring for the | Benzoin resin | Wheat germ oil | |
| skin | Bergamot oil | Avocado oil | |
| Regenerating | Geranium oil | Aloe vera | |
| the skin | Manuka oil | Borage oil | |
| | Myrrh oil | Jojoba oil | |
| | Vetiver oil | Almond oil | |
| | | Shea butter | |
| | | Dog rose | |
| Cardiotonic | | Arnica flowers | |
| | | Hawthorn extract | |
| Causing | Cassia oil | Arnica oil | Nicotine salicylate |
| hyperemia | Birch oil | Peanut oil | Capsaicin |
| Favoring the | Ginger oil | Olive oil | Capsaicinoids |
| blood flow | Camphor oil | | Caffeine |
| | Pine oil | | Benzyl nicotinate |
| | Oregano oil | | Nonivamide |
| | Black pepper oil | | Nicobexil |
| | Rosemary oil | | Methyl salicylate |
| | Savin oil | | |
| | Vetiver oil | | |
| | Eucalyptus oil | | |
| | Turpentine oil | | |
| | Camphor | | |
| | Silver fir oil | | |
| | Cinnamon oil | | |
| Immuno- | Camphor oil | Extracts from Stipites | |
| modulating | Cinnamon oil | Dulcamarae | |
| | Lemongrass oil | Viola tricolor | |
| | Melissa oil | Similax species | |
| | Niaouli oil | Phytolacca americana | |
| | Oregano oil | Glycyrrhiza glabra | |
| | Patchouli oil | Mistletoe extract | |
| | Tea tree oil | Bryonia alba | |
| | Thyme oil | Echinacea extract | |
| | Frankincense oil | | |
| Alleviating | Lavender oil | Melilotus officinalis | Bufexamac |
| itching | Manuka oil | Ruscus amleatus | Synthetic tannins |
| | | Fructus Capsici | Glycyrrhetinic acid |
| | | Capsicum (capsaicin) | |
| | | Borage oil | |
| | | Avocado oil | |
| | | Evening primrose oil | |
| | | Dog rose oil | |
| | | Tannins, e.g. from | |
| | | Quercus | |
| | | Hamamelis extract | |
| Keratolytic | | Mahonia aquifolium | Vitamin A acid |
| Antipsoriatic | | | Urea |
| | | | Salicylic acid |
| | | | Tazarotene |
| Cooling | Peppermint oil | | Menthol |
| Antimitotic | | | Colchicine |
| | | | Colchicine derivatives |
| Muscle relaxant | | | Peripheral, e.g.: |
| | | | Stabilizing: |
| | | | Tubocurarine |
| | | | chloride |
| | | | Alcuronium |
| | | | chloride |
| Continuation: | | | Preventing |
| Muscle relaxant | | | depolarization: |
| | | | Pancuronium |
| | | | bromide |
| | | | Vecuronium |
| | | | bromide |
| | | | Atracurium besylate |
| | | | Mivacurium |
| | | | chloride |
| | | | Rocuronium |
| | | | bromide |
| | | | Cisatracurium |
| | | | besylate |
| | | | Repolarizing, e.g.: |
| | | | Suxamethonium |
| | | | chloride |
| | | | Reduction of elevated |
| | | | skeletal muscle tone: |
| | | | Dentrols |
| | | | Irreversible inhibition |
| | | | of neuromuscular |
| | | | transmission: |
| | | | Clostridium |
| | | | Botulinum |
| | | | Botulin and |
| | | | derivatives |
| | | | Cotylinum (botox) |
| | | | Sodium channel |
| | | | inhibitors, such as |
| | | | tolperisone |
| | | | Local anesthetics |
| | | | Quinine sulfate |
| Caring for | Basil oil | Hamamelis extracts | Spartine sulfate |
| varicose veins | Cajeput oil | Ruscus aculeatus | Digitoxin |
| | Geranium oil | Melilotus albus | Heparin |
| | Melissa oil | Red vine leaf | Ergot alkaloids, in |
| | Niaouli oil | Aesculus | particular |
| | Tansy oil | hypocastanum | dihydroergotamine |
| | Tea tree oil | Melilotus officinalis | Diosmin |
| | Lemon oil | Centella extract | Flavonoid derivatives |
| | Cypress oil | Fagopyrum | |
| | | esculentum | |
| | | Pinus maritima | |
| Scale-inhibiting | Borage oil | | |
| | Evening | | |
| | primrose oil | | |
| Sedating | | Extractum Valerianae | |
| | | Melissa oil | |
| Spasmolytic | Peppermint oil | | |
| | Camphor oil | | |
| | Fir needle oil | | |
| Vasodilative | Birch oil | Hawthorn extract | Nitroglycerin |
| | Wintergreen oil | | Benzyl nicotinate |
| Healing | Bergamot oil | Dog rose | |
| wounds | Galbanum oil | Shea butter | |
| Antitraumatic | Geranium oil | Olive oil | |
| | Rose of Sharon | Evening primrose oil | |
| | oil | Arnica oil | |
| | Camomile oil | Avocado oil | |
| | Thuja oil | Aloe vera | |
| | | Jojoba oil | |
| | | Calendula oil | |
| | | Camomile oil | |
| | | Hamamelis extract | |
| | | Hypericum oil | |
| | | Tannins | |
| | | Calendula extract | |
| | | Symphytum extract | |
| | | Hypericum extract | |

By dissolution or dispersion of the abovementioned base materials, essential oils, plant extracts and/or synthetic single substances in a microemulsion, it is possible, inter alia, to formulate the following medicaments:

Medicaments for the treatment of external rheumatic pain which exhibit medicinal substances with an analgesic, antiinflammatory, hyperemia-causing and/or spasmolytic effect.

Medicaments for the treatment of complex peripheral pain syndrome which exhibit medicinal substances with an analgesic, antioxidant, antiinflammatory, spasmolytic, muscle-relaxing, hyperemia-causing and/or local anesthetic effect.

Medicaments for the treatment of wounds, contusions, strains, sports injuries and edemas which exhibit medicinal substances with a wound-healing, analgesic, thrombolytic, fibrinolytic, epithelizing, anticoagulant, antiinflammatory, antibacterial, antiviral, antimycotic, diuretic, skin-nourishing and/or antitraumatic effect.

Medicaments for the treatment of chronic wounds which exhibit medicinal substances with an antioxidant, analgesic, antiinflammatory and/or healing effect.

Medicaments for the treatment of hair loss.

Medicaments for the treatment of erectile dysfunction.

Medicaments for the treatment of excess secretion of sweat.

Medicaments for the treatment of neuralgia which exhibit medicinal substances with an analgesic and/or local anesthetic effect.

Medicaments for the treatment of diabetic neuropathy which exhibit medicinal substances with an analgesic, hyperemia-causing, alleviating of itching and/or alleviating of burning effect.

Medicaments for the treatment of varicosis or phlebitis which exhibit medicinal substances with a caring for varicose veins, protecting from edema, alleviating of itching, anticoagulant, fibrinolytic, antispasmodic, diuretic, deblocking, antioxidant and/or hemolytic effect.

Medicaments for the treatment of hemorrhoids which exhibit medicinal substances with a caring for varicose veins, diuretic and/or epithelizing effect.

Medicaments for the treatment of acute attacks of gout which exhibit medicinal substances with an antimitotic, antiinflammatory, antioxidant and/or diuretic effect.

Medicaments for the treatment of mycosis which exhibit medicinal substances with an antimycotic effect.

Medicaments for the treatment of neurodermatitis and/or eczema which exhibit medicinal substances with an antiinflammatory, alleviating of itching, immunomodulating, skin-regenerating, antioxidant, astringent and/or antiallergic effect.

Medicaments for the treatment of keratosis which exhibit medicinal substances with a keratolytic effect.

Medicaments for the treatment of psoriasis which exhibit medicinal substances with a keratolytic, antiinflammatory, alleviating of itching, skin-regenerating and/or antioxidant effect.

Medicaments for the treatment of acne which exhibit medicinal substances with a keratolytic, antibacterial, antiinflammatory, antioxidant and/or wound-healing effect.

Medicaments for the treatment of viral infections which exhibit medicinal substances with an antiviral, analgesic, antiinflammatory, keratolytic and/or antioxidant effect.

Medicaments for the treatment of hematomas which exhibit medicinal substances with a fibrinolytic effect.

Medicaments for the treatment of rosacea which exhibit medicinal substances with an antiinflammatory and/or antioxidant effect.

Medicaments for the treatment of scabies which exhibit medicinal substances with an antiparasitic and/or alleviating of itching effect.

Medicaments for the treatment of degenerated skin which exhibit medicinal substances with an antiinflammatory, antimicrobial, nourishing and/or local anesthetic effect.

Medicaments for the treatment of angina pectoris or chest pains which exhibit medicinal substances with a hyperemia-causing and/or spasmolytic effect and medicinal substances which interrupt pain stimuli.

Medicaments for the treatment of pruritus which exhibit medicinal substances with a cooling, local anesthetizing, analgesic, antiinflammatory and/or astringent effect.

Medicaments for the treatment of scars and keloids which exhibit medicinal substances which regulate connective tissue.

In a particularly preferred embodiment, several medicaments based on the same microemulsions can be combined to give combination preparations.

The concentration of the medicinal substances in the microemulsions results from the recommended guidelines of the therapy and the amount of microemulsion which can be handled in practice.

In concrete terms, the concentration of the medicinal substance in the microemulsion can be between 0 and 100%, concentrations between 10⁻⁸% and 50% being preferred and concentrations between 10⁻⁶ and 5% being particularly preferred.

Medicaments according to the invention for percutaneous administration are obtained by enriching, with oxygen, these and other medicaments based on microemulsions. This enriching can take place in the preparation of the medicinal substances.

The term “microemulsions enriched with oxygen” is understood to mean microemulsions which are enriched with oxygen in a suitable processing stage. Such a processing stage is represented, for example, by the atomization of the microemulsion in an oxygen-comprising atmosphere. In this connection, the oxygen content of this atmosphere is preferably greater than 25 percent by volume, particularly preferably greater than 50 percent by volume and in particular greater than 90 percent by volume.

Preferably, the microemulsion enriched with oxygen exhibits an oxygen concentration of greater than 10⁻³ mol/l, in particular of greater than 5×10⁻³ mol/l.

In order to prevent microemulsions enriched with oxygen in the preparation from re-releasing the oxygen up to the time of application, these microemulsions are preferably packaged in gastight containers.

In addition, other additives to these medicaments, and to other medicaments based on microemulsions, which improve the oxygen supply of the skin, result in medicaments according to the invention.

Examples of additives which improve the oxygen supply of the skin are natural oxygen carriers, such as myoglobin and/or hemoglobin, and also fluorocarbons.

An enriching of the microemulsion with oxygen can also be carried out directly in the administration of the microemulsion with the help of an application system for the percutaneous administration of medicinal substances exhibiting at least one microemulsion comprising medicinal substance and a device for the atomization of the microemulsion. In this connection, enriching with oxygen directly in the administration is preferred.

In such a system according to the invention, the microemulsion is preferably present in a container which is connected to an atomizing unit, a gas source under pressure being connected to the atomizing unit, and the microemulsion is atomized through the action of the pressurized gas.

It is likewise possible to at times abolish the barrier function of the stratum corneum by application of a microemulsion according to the invention without medicinal substances which is enriched with oxygen and/or which exhibits an additive which improves the oxygen supply of the skin. This also succeeds by application of a suitable microemulsion without medicinal substances, for example with an application system according to the invention. The medicinal substances to be administered are then applied to the relevant part of the skin in an additional stage.

On employing the system according to the invention, an oxygen-comprising propellant gas being used, the microemulsions which are applied are enriched with oxygen directly before the entry thereof into the stratum corneum. This results in an increase in the oxygen partial pressure on the tissue side of the stratum corneum and accordingly in stimulation of the cutaneous microcirculation and in improved transdermal transport of the medicinal substances. Likewise, the transdermal transport of medicinal substances can, for example, also be partly caused by an increased transmembrane pressure, here caused by the increase in the oxygen concentration on the tissue side of the stratum corneum.

The use of this system is particularly suitable with medicaments which exhibit substances sensitive to oxidation and which accordingly can be enriched with oxygen only directly before application.

It is possible, with an application system according to the invention, to accurately dose the dose of medicinal substance which is to be applied, through which the maximum daily dose can then also be applied. For that, a microemulsion which exhibits the maximum daily dose of one or more medicinal substances is sent into the system for the percutaneous administration of medicinal substances and is administered with this system to a patient.

An additional effect of the atomizing, which can contribute to improved transdermal transport of medicaments, is the spreading effect. This is based on the fine distribution of the droplets in the atomization. As a result, the microemulsion in the form of small droplets is more effective in falling into depressions, folds and openings in the skin.

The abovementioned medicaments based on microemulsions form preferred embodiments of a system for the percutaneous administration of medicinal substances in the context of this invention.

The application system according to the invention for the atomizing of liquid medicaments for the percutaneous administration of medicaments is explained more fully subsequently.

The implementation of the application system takes place according to the invention with the characteristics given in the patent claims.

In the application system according to the invention for the atomizing of liquid medicaments for the percutaneous administration of medicaments, a precisely dosed liquid medicament, in particular a microemulsion comprising the medicinal substance, for application to the skin by means of a propellant gas, preferably highly concentrated oxygen, is squeezed under pressure through a microdosing nozzle and is as finely atomized as possible, preferably through use of a suction action established through the Venturi effect.

A spectrum of droplet sizes can be generated with the microdosing nozzle of the application system, the outlet cross section of the microdosing nozzle being varied by a positionable needle point and accordingly it being possible to change the droplet size. The diameter of the droplets which can be obtained by the atomizing lies in the nanometer range, the mean droplet size measured being less than 1 μm, preferably less than 400 nm, in particular less than 300 nm. The reproducibility of the spectrum of droplet sizes with the application system can be demonstrated by noncontact measuring methods using laser optics.

From the multitude of the different droplets of an atomization liquid, the individual droplet sizes and the frequency thereof can be determined using laser diffraction spectroscopy. In this connection, the monochromatic light of a laser beam is diffracted more or less strongly by the individual droplets of an atomization liquid, the photomultipliers located on a detector registering different signals and intensities. In line electronics with specific software evaluate these and calculate from this the actual droplet size distribution.

All liquid medicaments prepared and to be atomized, preferably medicaments based on microemulsions, with particular rheological properties, such as, e.g., viscosity, liquid density, surface tension but in particular below a certain dynamic viscosity, can be sprayed onto the site of the skin to be treated using the application system according to the invention.

Apart from highly concentrated oxygen, air, nitrogen or a noble gas (helium, argon) can alternatively be used as propellant gas. In this connection, the term “highly concentrated oxygen” is understood to mean a gas which is enriched with at least 90% by volume of oxygen. If propellant gases are used which comprise no oxygen, the microemulsion is already enriched with oxygen and/or comprises additives which improve the oxygen supply of the skin.

In the atomizing, the medicament prepared is surrounded by propellant gas and mixed with this. In this connection, the propellant gas dissolves in the liquid medicament under pressure, through which a positive property of the liquid active substance stimulating the skin in connection with oxygen can be produced.

A positive effect of the extremely fine atomizing is the pleasantly cooling action, because of the cold due to evaporation, of the finely atomized medicament in the percutaneous administration of medicaments.

Because of the reactivity of the highly concentrated oxygen, materials which withstand oxygen are to be used for the individual components of the application system, such as, e.g., glass, special hospital-grade plastics or high-grade steel.

In the atomizing of the microemulsion, it is advantageous to achieve, depending on the daily dose and body part to be treated, a volumetric flow rate of 1.5 to 5 ml/20 min or 4.5 to 15 ml/h through the outlet cross section of the microdosing nozzle.

The propellant gas can be withdrawn from a gas container and can be conveyed to the application system via a hose connection. The gas container itself can be a constituent of an oxygen preparation plant (O₂ plant), in which oxygen is obtained from ambient air and is enriched in this.

Alternatively, in an additional embodiment of application system and gas source, a self-sufficient gas container or a gas connection is also conceivable in a clinic.

In a preferred embodiment of the invention, the application system is in the form of a self-sufficient system filled with liquid medicament and connected to a propellant gas system.

The application system according to the invention for the percutaneous administration of medicinal substances, in particular of liquid medicaments based on microemulsions, is more fully explained below with reference to FIGS. 1, 2 and 3.

### BRIEF DESCRIPTION OF THE DRAWINGS

FIG. 1 shows a diagrammatic representation of an application system,

FIG. 2 shows an enlarged diagrammatic representation of the region of the application system according to FIG. 1 in the vicinity of the nozzle and the operating principle thereof, and

FIG. 3 shows a diagrammatic representation of an additional application system,

FIG. 4 shows a diagrammatic representation of an additional application system.

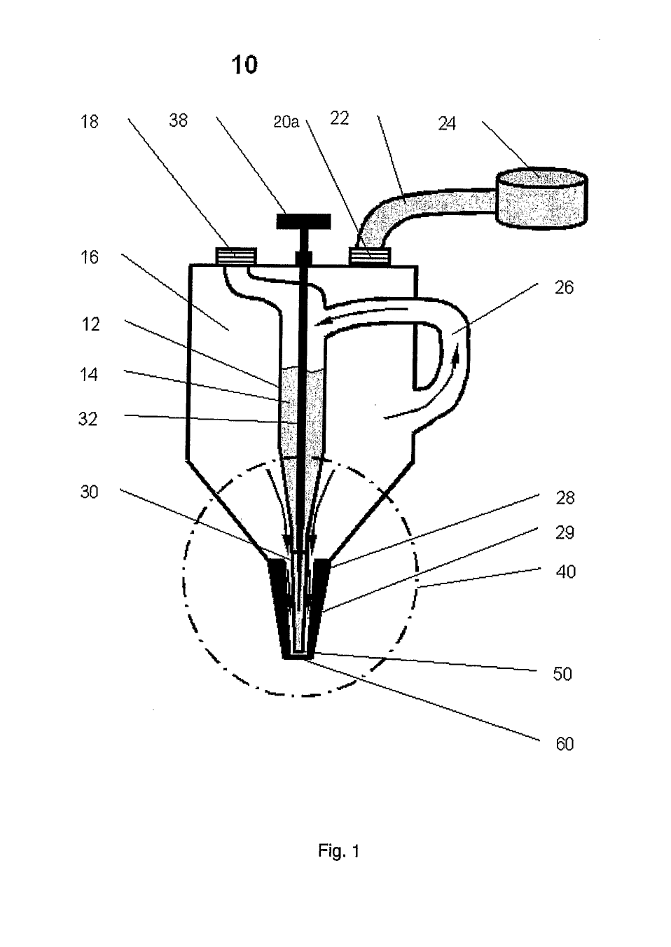

FIG. 1 shows an application system 10 in simplified diagrammatic representation of the individual components. The application system comprises a medicament reservoir 12 which is arranged in a gas reservoir 16 of the application system 10. The medicament reservoir 12 is tapered at its end in the region 40 of the application system 10 in the vicinity of the nozzle to give a capillary. Depending on the daily dose to be administered, between 1.5 and 5 ml of a medicament 14 are located in the medicament reservoir 12. The upper end of the medicament reservoir 12 and the gas reservoir 16 of the application system 10 are in the normal position seen to be coaxially formed and are connected to one another via a bypass line 26 or an equalizing pipe 26. An inlet 18 for filling the medicament reservoir 12 with a medicament 14 and an inlet 20 for filling the gas reservoir 16 with a propellant gas are likewise located at the upper end. The gas reservoir 16 of the application system 10 is connected via a hose connection 22 to a gas container 24. In the region 40 in the vicinity of the nozzle, the application system 10 has the form of a solid of rotation with a cross section tapering in the direction of the nozzle outlet 50. The medicinal substance reservoir 12 connects with its tapered end to the atomizing nozzle 30, which is arranged inside the nozzle head 28. The nozzle head 28 exhibits, along its axis of rotation, openings 29 via which the gas reservoir 16 is connected flow wise with the surroundings. A needle 32 carried in the upper part of the gas reservoir 16 projects into the atomizing nozzle 30 and narrows the annular cross section thereof. The needle can be vertically positioned by turning a knurled head 34 and the narrowing of the cross section of the atomizing nozzle 30 can thereby be adjusted.

The manner of operation of the application system 10 represented in FIG. 1 for the atomizing of a prepared medicament for the percutaneous administration of medicaments is more fully described below.

Depending on the size of the area of the body part to be treated, the medicament reservoir 12 is filled, via the medicament reservoir inlet 18 of the application system 10, with a precisely dosed liquid medicament 14, in particular a liquid medicament based on a microemulsion, preferably from 1.5 to 5 ml.

For the atomizing of the liquid medicament 14, the gas reservoir 16 is continuously filled with propellant gas, preferably oxygen, through which an excess pressure builds up in the closed gas reservoir 16. The propellant gas is withdrawn from the gas container 24 and conveyed to the application system 10 under a predetermined pressure, in the example approximately 2 bar. For this, the gas reservoir 16 is connected via a hose connection 22 to a gas connection 20 of the application system 10.

The propellant gas is transported, by the excess pressure in the gas reservoir 16, up to the outlet 50 of the atomizing nozzle 30 (microdosing nozzle). Since the gas reservoir 16 of the application system 10 in the region 40 in the vicinity of the nozzle has the form of a solid of rotation with a cross section tapering in the direction of the nozzle outlet 40, the propellant gas is accelerated by the excess pressure in the gas reservoir 16 in the flow direction. The dynamic pressure appearing inside the gas reservoir 16 as a result of the narrowing in the cross section is diverted via a bypass line 26 to bring about the advance of the liquid medicament 14 in the medicament reservoir 12, the dynamic pressure squeezing the liquid medicament through the atomizing nozzle 30. A uniform advance is provided by this.

The end of the medicament reservoir tapering in the region 40 in the vicinity of the nozzle inside the gas reservoir 16 is shaped in such a way that the liquid medicament is prevented from breaking off.

The openings 29 inside the nozzle head 28 guarantee that the propellant gas accelerated in the direction of the tapering solid of rotation 16 flows around the atomizing nozzle 30 up to the outlet 50 of the nozzle head 28.

Having arrived at the outlet 50 of the atomizing nozzle 30, the liquid medicament is sucked in by the negative pressure appearing in the outlet (Venturi effect) and is at the same time atomized.

In the atomizing, the prepared medicament 14 is surrounded by the propellant gas and is mixed with this. In this connection, the propellant gas dissolves in the liquid medicament 14. This results in a strengthened effect of the liquid medicament 14 on the microcirculation, in particular in a liquid medicament based on microemulsions, which can result in percutaneous administration of medicinal substance. The droplet size diameter in the atomizing of the liquid medicament 14 can be varied via the needle 32 inside the atomizing nozzle 30, by finely positioning the needle 32 by turning the knurled head 38. If the atomizing nozzle 30 is completely closed by the needle 32, so that the mass flow of the liquid medicament 14 through the atomizing nozzle 30 is prevented, the atomizing of the medicament comes to a standstill. Then simply propellant gas flows through the outlet 60 of the nozzle head 28, because of the openings 29 arranged inside the nozzle head 28 along the atomizing nozzle 30.

On the other hand, in an additional embodiment not represented, a nozzle with a predetermined internal diameter without an adjusting needle can be used if through this the desired droplet profile is already achieved.

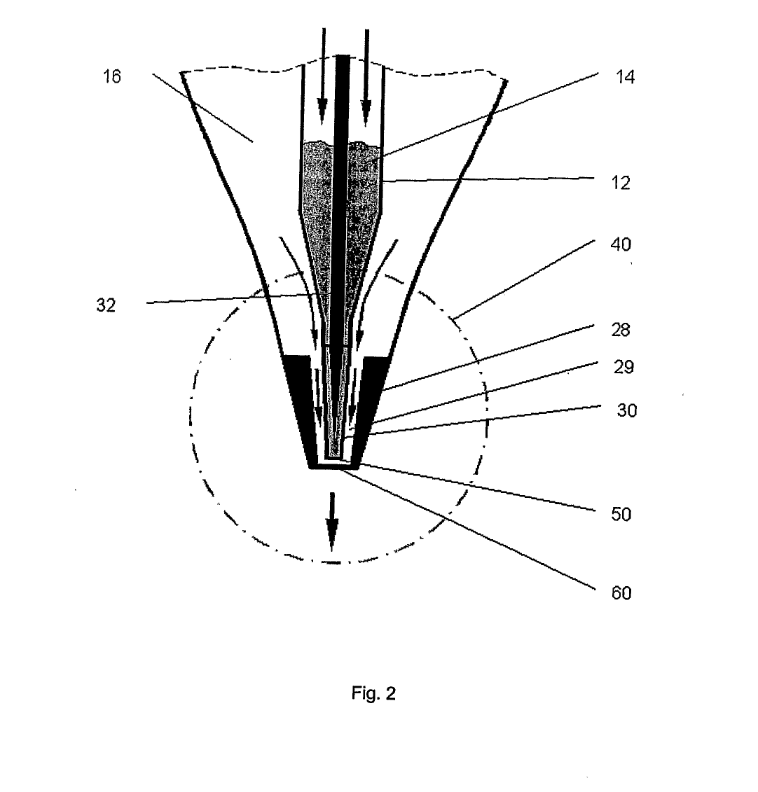

FIG. 2 shows the operating principle of the atomizing represented diagrammatically in simplified form in FIG. 1, the region 40 of the application system 10 in the vicinity of the nozzle being represented for clarification on an enlarged scale. In this connection, the arrows indicate the direction of flow of the gas.

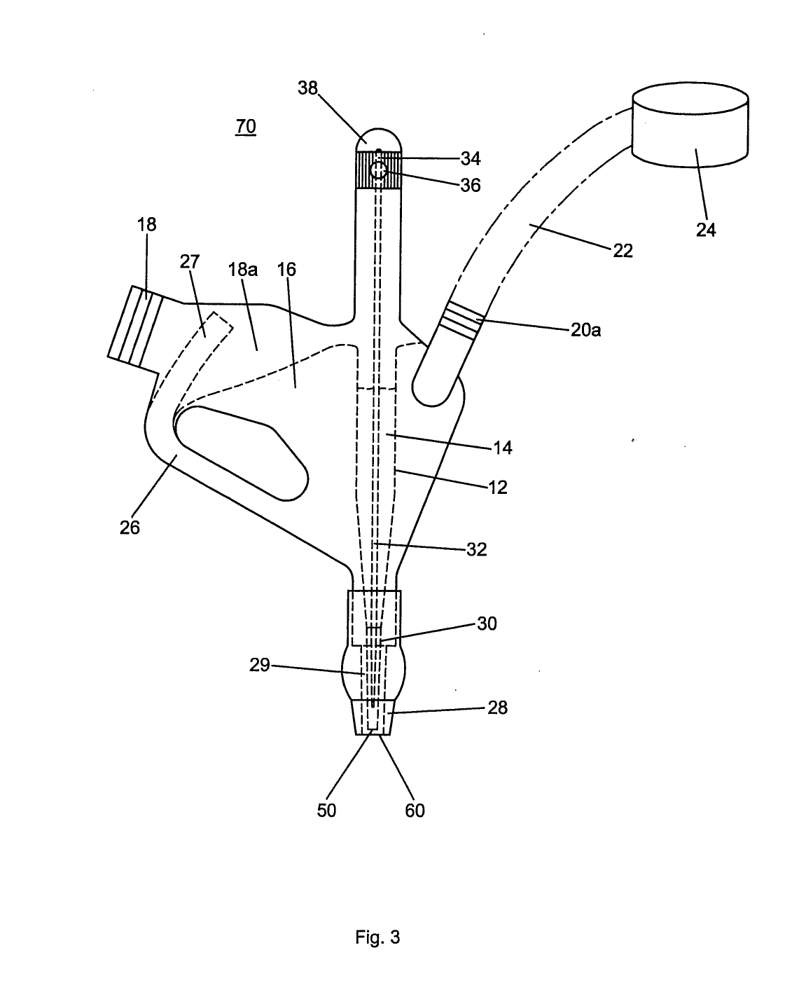

FIG. 3 shows an additional exemplary embodiment of an application system 70 in cross section. The application system 70 comprises a medicament reservoir 12 which is surrounded by a gas reservoir 16 of the application system 70. The medicament reservoir 12 is, at its end in the region of the application system 70 in the vicinity of the nozzle, shaped or tapered to give a capillary. Depending on the daily dose to be administered, between 1.5 and 5 ml of a medicament 14 are located in the medicament reservoir 12. The medicament reservoir 12 and the gas reservoir 16 of the application system 70 are formed coaxially and are connected to one another via a bypass line 26. A medicament reservoir inlet 18, for filling with a medicament 14, and a gas reservoir inlet 20, for filling the gas reservoir 16 with propellant gas, are located on the upper end of the application system 70. Both inlets can be closed by caps, not shown.

The medicament reservoir inlet 18 is shaped in such a way that the liquid medicament 14 can in no case reach the bypass 26 and accordingly run out from the application system 70. In order to prevent this, the bypass end 27 was shaped in such a way that it projects far into the inlet line of the medicament reservoir 12.

The gas reservoir 16 of the application system 70 is connected via a hose connection 22 to a gas container 24. In the region in the vicinity of the nozzle, the application system 70 has the form of a solid of rotation with a cross section tapering in the direction of the nozzle outlet 50. The medicinal substance reservoir 12 connects with its tapered end to the atomizing nozzle 30, which is arranged inside the nozzle head 28. The nozzle head 28 exhibits, along its axis of rotation, recesses 29 so that the gas reservoir 16 is connected flow wise with the surroundings. A needle 32 carried in the upper part of the application system projects into the atomizing nozzle 30 and narrows the annular cross section thereof. The needle 32 can be positioned vertically by turning the adjustable screw (knurled screw) arranged in the knurled head 34 and through this the narrowing in cross section of the atomizing nozzle 30 can be adjusted.

The manner of operation of an additional application system 70, represented in FIG. 3, for the atomizing of a prepared medicament for the percutaneous administration of medicaments is described more fully below.

Depending on the size of the area of the part of the body to be treated, the medicament reservoir 12 is filled, via the medicament reservoir inlet 18 of the application system 70, with a precisely dosed medicinal substance 14, in particular in a microemulsion, preferably from 1.5 to 5 ml.

For the atomizing of the liquid medicament 14, the gas reservoir 16 is continuously filled with propellant gas, preferably oxygen, through which an excess pressure builds up in the gas reservoir 16. The propellant gas is withdrawn from a gas container 24 and conveyed to the application system 70 under a predetermined pressure. For this, the gas reservoir 16 is connected via a hose connection 22 to a gas connection 20 of the application system 70.

The propellant gas is transported, by the excess pressure in the gas reservoir 16, up to the outlet 50 of the atomizing nozzle 30 (microdosing nozzle). Since the gas reservoir 16 of the application system 70 in the region 40 in the vicinity of the nozzle has the form of a solid of rotation with a cross section tapering in the direction of the nozzle outlet 40, the propellant gas is accelerated by the excess pressure in the gas reservoir 16 in the flow direction. The dynamic pressure appearing inside the gas reservoir 16 as a result of the narrowing in the cross section is diverted via a bypass line 26 to bring about the advance of the liquid medicament 14 in the medicament reservoir 12, the dynamic pressure squeezing the liquid medicament through the atomizing nozzle 30. A uniform advance is provided by this.

The end of the medicament reservoir 12 inside the gas reservoir 16, which end is shaped in the region in the vicinity of the nozzle as an internal capillary, is shaped in such a way that the liquid stream 14 is prevented from breaking off. The recesses 29 inside the nozzle head 28 guarantee that the propellant gas accelerated in the direction of the tapering solid of rotation 16 flows around the atomizing nozzle 30 up to the outlet 50 of the nozzle head 28.

Having arrived at the outlet 50 of the atomizing nozzle 30, the liquid medicament is sucked in by the negative pressure appearing in the outlet (Venturi effect) and is at the same time atomized.

The droplet size diameter in the atomizing of the liquid medicament 14 can be varied via the needle 32 inside the atomizing nozzle 30, by finely positioning the needle 32 by turning the knurled screw 36 arranged in the knurled head 38. If the atomizing nozzle 30 is completely closed by the needle 32, so that the mass flow of the liquid medicament 14 through the atomizing nozzle 30 is prevented, the atomizing of the medicament comes to a standstill. Then simply propellant gas flows through the outlet 60 of the nozzle head 28, because of the recesses 29 arranged inside the nozzle head 28 along the atomizing nozzle 30.

The conicity of the needle 32 is more strongly developed in comparison with the conicity of the atomizing nozzle 30 for the purposes of a broader atomizing or a broader atomizing angle.

A broader atomizing angle can furthermore be pursued by the incorporation in the nozzle head 28 of a helix-producing means.

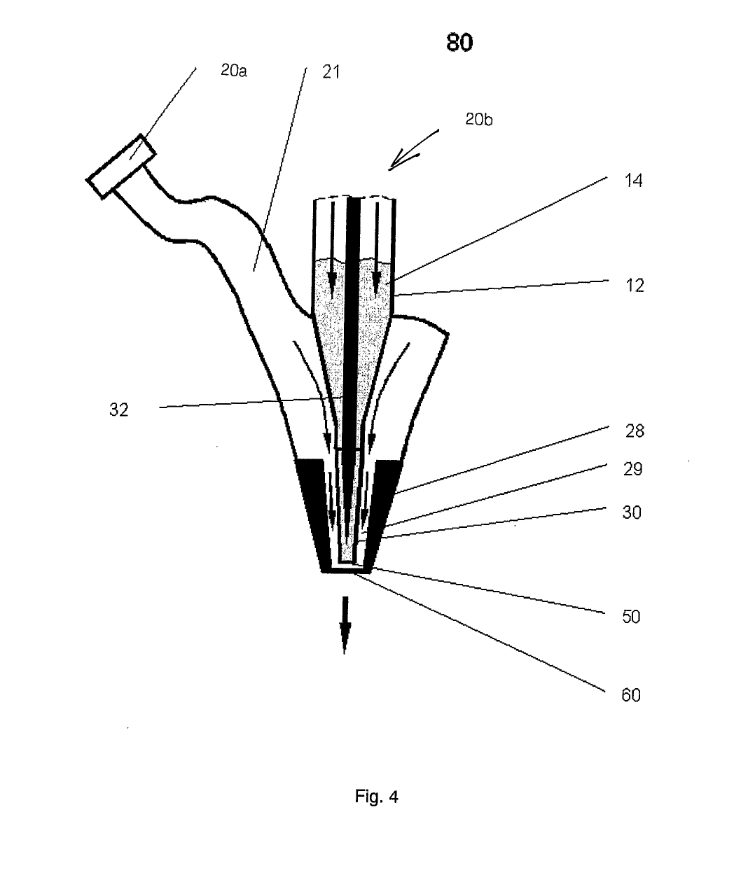

According to an additional embodiment—shown in FIG. 4—the medicament reservoir is combined on its upper side directly with the gas source and accordingly has an additional inlet. In this connection, the Venturi formation of the nozzle can be dispensed with if this appears advisable. If, however, Venturi atomizing nozzle is used, a gas supply arrangement corresponding to FIG. 2 is provided on the outside of the nozzle. A gas reservoir can thus be dispensed with except for the region of the nozzle, if only a gas supply in the region of the nozzle is provided, as is represented in FIG. 4.

Microemulsions and the preparation thereof are described below from examples, which microemulsions can in the context of this invention be enriched with oxygen, for example in the administration in the application system according to the invention. These examples are not to have a limiting effect.

### Example 1

### Manufacture of a Water-in-Oil Microemulsion (I)

5 g of Tween® 80 are mixed with 10 g of Span® 20 and 5 g of ethanol, and 75 g of isopropyl myristate are added. 5 g of water are added dropwise to this mixture with stirring. This gives 100 g of a water-in-oil microemulsion (I).

### Example 2

### Manufacture of a Water-in-Oil Microemulsion (II)

14 g of Span® 20 are mixed with 21 g of Synperonic® PEL 101.60 g of isopropyl palmitate are added thereto. 5 g of water are added dropwise to this mixture with stirring. This gives 100 g of a water-in-oil microemulsion (II).

### Example 3

### Manufacture of an Oil-in-Water Microemulsion (III)

4 g of Tween® 80 are mixed with 12 g of Synperonic® PEL 101.5 g of isopropyl myristate are added thereto. 79 g of a water/polypropylene glycol (1:2) (weight ratio) mixture are added to this mixture with stirring. This gives 100 g of an oil-in-water microemulsion (III).

### Example 4

Preparation of a medicament with the medicinal substance procaine, for the local combating of pain, based on an oil-in-water microemulsion: 2 g of procaine chloride are dissolved in 5 ml of water. The solution is added to 93 g of the microemulsion III with stirring. This gives 100 g of the medicament.

### Example 5

Preparation of an additional medicament with the medicinal substance procaine, for the local combating of pain, based on a water-in-oil microemulsion:

2 g of procaine chloride are dissolved in 5 g of 0.01M NaOH. The solution is added dropwise with stirring to 93 g of the microemulsion I. This gives 100 g of the medicament.

### Example 6

Preparation of a medicament with the medicinal substance lidocaine, for the local combating of pain, based on a water-in-oil microemulsion: 2 g of lidocaine are dissolved in 98 ml of the microemulsion II. This gives 100 g of the medicament.

### Example 7

Preparation of a medicament with the medicinal substance diclofenac, for the local combating of painful inflammation, based on a water-in-oil microemulsion:

2 g of lidocaine, 2 g of diclofenac and 0.05 g of capsaicin are successively dissolved in 95.95 g of the microemulsion (II). This gives 100 g of the medicament.

1. (canceled)

2. A method for percutaneously delivering a medicinal substance to a patient in need thereof, comprising:
a) providing a microemulsion that comprises at least one medicinal substance for percutaneous administration;
b) dispersing the microemulsion into droplets using a pressurized gas comprising oxygen, thereby producing an atomized microemulsion that is enriched with oxygen; and
c) applying the atomized microemulsion that is enriched with oxygen to the skin of the patient, wherein the medicinal substance penetrates the horny layer of the skin, thereby percutaneously delivering the medicinal substance to the patient.

3. The method of claim 2, wherein the droplets range in size from about 10 nm to about 1 μm.

4. The method of claim 3, wherein the mean size of the droplets is less than 150 nm.

5. The method of claim 2, wherein the pressurized gas has an oxygen content that is greater than 25% by volume.

6. The method of claim 5, wherein the oxygen content is greater than 50% by volume.

7. The method of claim 6, wherein the oxygen content is greater than 90% by volume.

8. The method of claim 2, wherein the microemulsion is an oil-in-water emulsion.

9. The method of claim 2, wherein the microemulsion is a water-in-oil emulsion

10. The method of claim 2, wherein the atomized microemulsion that is enriched with oxygen is sprayed onto the skin of the patient at a site to be treated.

11. The method of claim 2, wherein steps (b) and (c) are performed at the same time.

12. The method of claim 2, wherein the microemulsion is dispersed and applied to the skin of the patient using a device, comprising:
a medicament reservoir which comprises the microemulsion;
a first gas connection, by which gas can be conveyed under a predetermined pressure via a gas feed pipe into the medicament reservoir;
a medicament reservoir inlet, by which the microemulsion can be conveyed;
a nozzle head with recesses, which is arranged at the end of the medicament reservoir;
an atomizing nozzle, which is arranged in the nozzle head and is connected flow wise with the medicament reservoir, and by which a spectrum of droplet sizes can be generated, the nozzle head and the atomizing nozzle forming a Venturi arrangement, and the nozzle head exhibiting an annular space around the atomizing nozzle; and
a second gas connection, which is arranged in the region of the nozzle head and is connected flow wise via a gas feed pipe with the annular space and the recesses, the droplets generated by means of pressure at the outlet of the atomizing nozzle being atomized by the Venturi effect.

13. A method of treating pain in a patient in need thereof, comprising percutaneously delivering a medicinal substance for treating pain to the patient according to the method of claim 2.

14. A method of treating a circulatory disorder in a patient in need thereof, comprising percutaneously delivering a medicinal substance for treating the circulatory disorder to the patient according to the method of claim 2.

15. A method for treating a wound in a patient in need thereof, comprising percutaneously delivering a medicinal substance for treating the wound to the patient according to the method of claim 2.

16. A method for treating psoriasis in a patient in need thereof, comprising percutaneously delivering a medicinal substance having a keratolytic, antiinflammatory, alleviating of itching, skin-regenerating or antioxidant effect to the patient using the method of claim 2.

17. A method for administering a medicinal substance for percutaneous administration to a patient in need thereof, comprising the steps of:
a) providing a microemulsion that comprises at least one medicinal substance for percutaneous administration;
b) mixing the microemulsion with a propellant gas comprising oxygen under pressurized conditions; and
c) spraying the microemulsion onto the skin of the patient in the form of droplets ranging in size from about 10 nm to about 1 μm, wherein the medicinal substance penetrates the horny layer of the skin, thereby administering the medicinal substance percutaneously to the patient.
